(19) United States
(12) Patent Application Publication
(10) Pub. No.: US 2009/0277814 A1
(43) Pub. Date: Nov. 12, 2009

(54) SECURABLE EARPLUG PACKAGE

(75) Inventors: Michael E. Hamerly, Vadnais Heights, MN (US); Sharon R. Garber, Plymouth, MN (US); Lance E. Behymer, Woodbury, MN (US); Philip G. Martin, Forest Lake, MN (US)

Correspondence Address: 3M INNOVATIVE PROPERTIES COMPANY, PO BOX 33427, ST. PAUL, MN, 55133-3427, US

(73) Assignee: 3M Innovative Properties Company

(21) Appl. No.: 12/118,180

(22) Filed: May 9, 2008

(51) Int. Cl. B65D 85/00; A61F 11/08; A61F 11/00

(52) U.S. Cl. 206/526; 128/864

(57) ABSTRACT

An earplug assembly 10 that includes an earplug system 20 that has an earplug 12, 12′ and a package portion 32 such as a deformable sheath 14 that individually surrounds at least the body 31 of the earplug 12, 12′. A securement portion 28 is joined to the packaging 32 to enable the assembly 10 to be secured to another object such as a worker's helmet 30. In this manner, the earplugs can be readily available to the user in a protected condition. Opportunities for misplacement of the earplugs are reduced.

### BACKGROUND

Earplugs have been well accepted for use in providing sound attenuation to people. To protect the earplugs from contamination until use, the earplugs are commonly packaged in pairs in translucent plastic bags, in cardboard pillow-pack containers, or in plastic receptacles (see, for example, U.S. Pat. Des. 433,226 and 434,217 to Foslien). Earplug users typically will carry the earplug package with them until the earplugs are needed. Often the user will place the earplug package in their pocket until needed or will set it in a convenient location. Sometimes, however, the earplugs can be misplaced and therefore will not be readily available to the user when needed.

To make earplugs readily available to workers, earplug dispensers are commonly placed in central locations at the workplace. Examples of earplug dispensers are shown in U.S. Pat. Nos. 6,241,120, 5,954,229, and 5,280,845 and U.S. Publication 2002/0139707A1, International Publication WO 02/00155A2, and European Patent Application 1,074,236A2.

Although the earplug dispensers tend to be placed in a location that is convenient to the employees, this method of product distribution may nonetheless sometime require that the workers walk to and from the dispenser to obtain their earplugs; it also does not make the earplugs readily available in other locations, for example, when workers are out in the field. Further, earplugs that are distributed through dispensing machines typically are not protected by packaging. As such, the earplugs can be exposed to contaminants if placed in the user's pocket or if set aside for use at a later time.

### SUMMARY OF THE INVENTION

The present invention provides an earplug package that allows earplugs to be conveniently carried by the user and protected from contamination until use. The present invention provides these advantages through an earplug assembly that comprises (a) one or more earplugs disposed in a package; and (b) a means for securing the package to another object, the means for securing to another object being joined to the package.

The present invention is beneficial over known earplug packaging and distribution systems in that it includes the means for securing the earplug package to another object. The inventive earplug assembly thus can be secured to an article or device that is commonly worn, attached to, or carried by the user so that the earplugs are readily available to the user when needed. The inventive earplug assembly thus eliminates problems encountered in the workplace, including preventing the earplugs from becoming contaminated and misplacing them at a forgotten location.

### GLOSSARY

“body” means the general portion of the earplug that is fashioned for being disposed in the human ear canal during earplug use;

“compressed” means reduced in size through pressure application;

“deformable” means having the ability to be readily altered in shape by mere finger pressure;

“earplug” means a device that is designed for being inserted into the human ear canal to attenuate sound;

“earplug assembly” means a device for storing earplugs until use;

“earplug system” means a combination that includes one or more earplugs and a package for the earplug(s);

“foam earplug” means an earplug that has voids within a solid material to allow at least that body of the earplug to be compressed and then expanded from the compressed condition;

“individually surrounds” and “individually disposed in” means located about a single one (or a portion thereof)—not two or more—of the items in question;

“object” means a tangible thing;

“package” means a preassembled unit fashioned for storing another object(s) such as earplug(s) until they are desired to be used;

“plastic” and “polymeric” means comprising polymers mainly, along with other possible ingredients;

“noncompressed condition” means not compressed from its relaxed state;

“separation line” means a cut, perforation, score, or tear which demarcates the place where two adjoining parts (e.g., sheaths) can be separated;

“sheath” means a structure that is designed to temporarily, and at least partially, surround another item until the item is removed therefrom.

### BRIEF DESCRIPTION OF THE DRAWINGS

FIG. 1 is a front view of an earplug assembly 10 in accordance with the present invention;

FIG. 2 is a side cross-sectional view taken along lines 2-2 of FIG. 1, showing a foam earplug 12 located in a sheath 14 in accordance with the present invention;

FIG. 3 is a top cross-sectional view taken along lines 3-3 of FIG. 2, showing a series of earplugs 12 disposed in a series of sheaths 14 in accordance with the present invention;

FIG. 4 is a front view of an earplug assembly 10′ in accordance with the present invention;

FIG. 5 is a front view of earplug assembly 10″ in accordance with the present invention; and

FIG. 6 is a perspective view of an earplug assembly 10 secured to a worker's helmet in accordance with the present invention.

FIGS. 7a and 7b are perspective views of earplug systems—that may be separated from an earplug assembly, in accordance with the present invention; and

FIG. 8 is a perspective view that shows how a person can compress an earplug 12 in a sheath 14 after removing this combination from an earplug assembly 10 of the present invention.

### DETAILED DESCRIPTION OF PREFERRED EMBODIMENTS

FIG. 1 shows an earplug assembly 10 that includes series of earplugs 12 that are individually disposed in a plurality of transparent packages, which may take the form of sheaths 14. Each sheath 14 may be separated from an adjoining sheath 14 by a seal line 16 such as a bond or weld line. Each sheath 14 also may have a separation line 18, which can be a perforation or score line. The separation line may be used in lieu of, in conjunction with, or be the same as the seal line and may be located between an adjoining sheath 14 to facilitate separation of an individual earplug system 20 from an adjoining earplug system. A user therefore can individually separate an earplug system 20 from the assembly 10 just prior to use. The earplug assembly 10 may contain two, four, or more earplug systems 20, and up to, for example, 10 to 20 earplug systems. The earplug assemblies also may be provided in bulk form, for example, in a roll that contains up to approximately 100, 200, or 300 individual earplug systems. The desired length of the assembly could be separated from the bulk supply along one of the separation lines noted above. This smaller assembly of earplug systems could then be carried by the worker or secured to an adjacent object until use.

FIG. 1 also shows that a second separation line 19 can be provided in each earplug sheath to enable the earplug 12 to be more easily removed from the sheath 14. The separation line 19 may be provided in the general region where the earplug body meets end 22. As the term is used in this document, “end” does not mean precisely the outer edge of the object but includes some portion extending inwardly. In this sense, the word “end” is used in its common sense—like the “south end of town” or the “front end of a car”.

FIG. 2 shows that the sheath 14 may completely enclose the foam earplug 12 until use. In this manner of packaging, the earplugs are fully protected from contamination. FIG. 2 also shows that an earplug assembly 10 may include an adhesive layer 22 and a release liner 24. The adhesive layer 22 allows the assembly 10 to be secured to a desired location. To secure the assembly 10 to another object, the user simply removes release liner 24 and presses the adhesive layer 22 against the object by applying a force at surface 25. The adhesive can be, for example, a pressure-sensitive adhesive. Classes of suitable pressure-sensitive adhesives include acrylics, tackified rubber, tackified synthetic rubber, ethylene vinyl acetate, silicone, and the like. The adhesive may be applied directly to the earplug assembly such as with a hot-melt adhesive coating process. The adhesive may also be applied in the form of a transfer tape, using for example, Scotch™ ATG adhesive Transfer Tape 924 from the 3M Company. The earplug assembly 10 may be adhered to another object worn or carried by the user so that the earplugs 12 are readily available for use. In lieu of an adhesive, a mechanical fastener could be used, for example, a hook and look type fastener. The earplugs 12 remain in a protective condition in each sheath 14 until the user enters an environment where the earplugs are needed. The user then can grasp an earplug system 20 from the object, separating it from the earplug assembly 10 at the separation line 18 (FIG. 1), and compressing the earplug 12 as illustrated, for example, in FIG. 8. The sheath 14 may completely surround each earplug 12 individually to prevent it from being contaminated by any dust or contaminants that may be present in the air. A second line of separation 19 may be provided in the sheath 14 to enable it to be easily opened for purposes of removing an earplug 12 prior to insertion into the ear canal. In lieu of an adhesive layer, the assembly may include a glue, a mechanical fastener such as a hook-and-loop device, or any other suitable physical or mechanical mechanism for securing the assembly to the object.

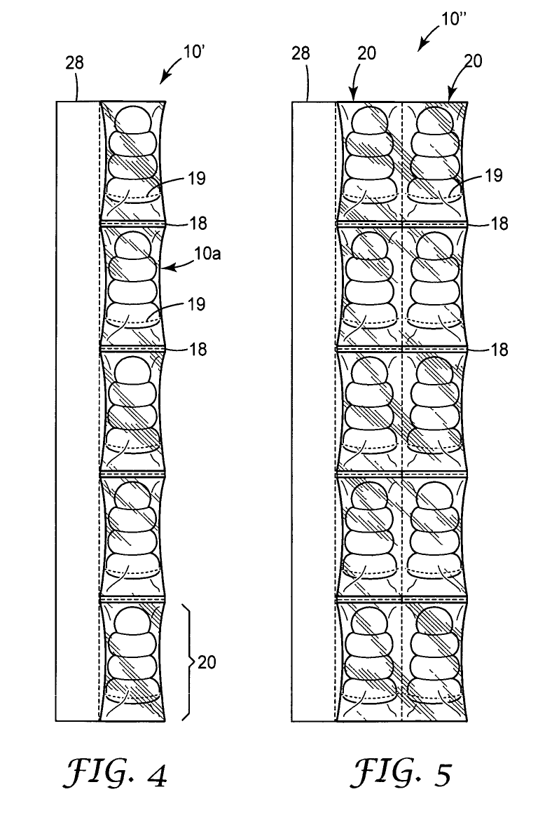

FIG. 4 illustrates an alternative arrangement of an earplug assembly 10′. In this embodiment, the earplugs are disposed in the same direction as the tape portion 28 of the assembly 10′. This embodiment may be preferred where a narrower earplug assembly is desired. Each earplug system—may be separated from an adjoining earplug system—by a perforation line 18, and an additional separation line 19 can be provided in each sheath to allow the earplug to be easily removed therefrom.

FIG. 5 shows yet another embodiment of an earplug assembly 10″. In this embodiment, the earplug systems 10″ are provided in matched pairs that are separated by separation lines 18 so that a pair of earplugs 12 can be conveniently removed at the same time. This embodiment also includes a tape 28 to allow the assembly 10″ to be adhered to another object such as a worker's helmet. As shown in the embodiments discussed above, the adhesive and release liner may be positioned on the assembly to extend lengthwise from a first edge to a second edge. When the release liner is removed and the assembly is adhered to another object, the earplug systems drape below the tape so that they can be easily grasped by the user.

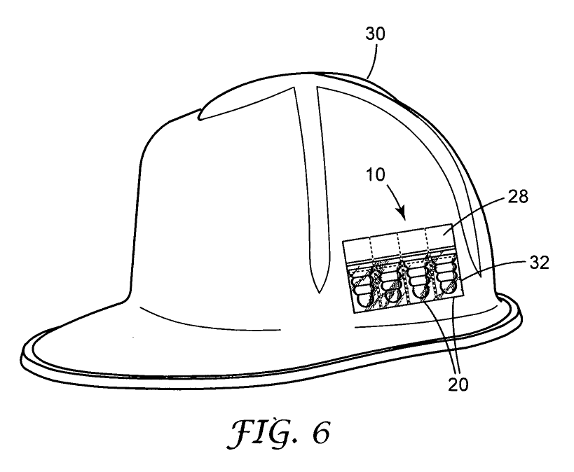

FIG. 6 shows the earplug assembly 10 secured to a helmet 30 in accordance with the present invention. The earplug assembly 10 is secured to the side of the helmet such that the adhesive portion 28 resides above the package portion 32. That is, the earplugs in the individual packages tend to drape beneath the adhesive strip. When a wearer of the helmet requires hearing protection, the wearer can grasp an individual earplug system 20 and separate it from adjoining earplug systems 20. The user can then compress the earplug within the sheath (FIG. 8), remove it from the sheath interior, and place it in their ear canal where it can expand to attenuate sound from the ambient environment. In lieu of a helmet 30, the earplug assembly 10 could be secured to other objects such as a worker's safety vest, a belt, a jacket, a respiratory harness, protective eyewear, a tool box, a noisy tool such as an air hammer, et cetera.

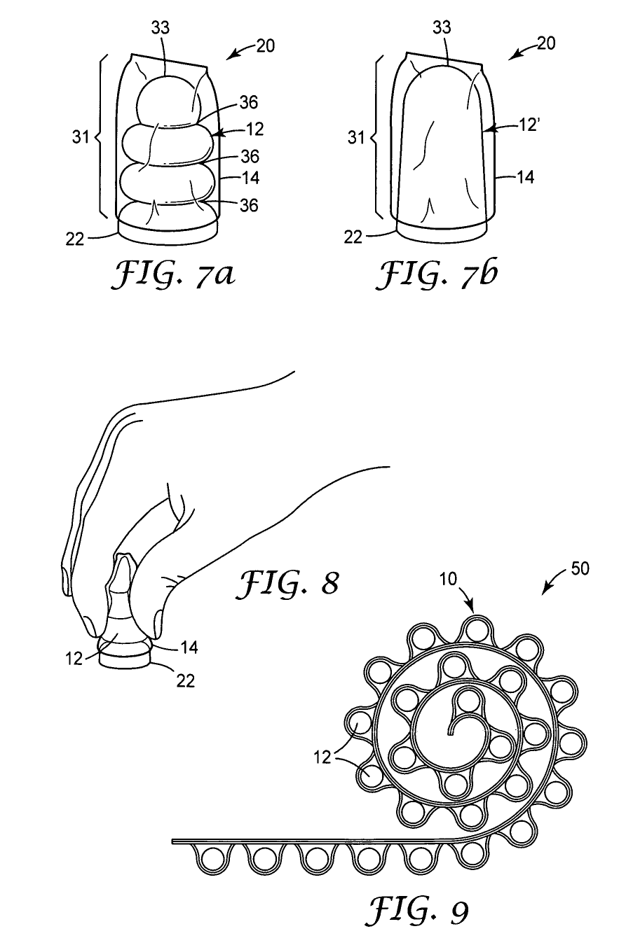

FIGS. 7a and 7b show an earplug system 20 that has a foam earplug 12, 12′, and a sheath 14. Each earplug 12 and 12′ has a body portion 31 and first and second ends 22 and 33. The body portion 31 is the general portion of the earplug 12, 12′ that would be inserted into the user's ear canal. Typically, the earplug end 22 protrudes from the ear canal during use so that the user can adjust the earplug or remove it from the ear canal as needed. The body portion 31 of earplug 12, as shown in FIG. 7a, can have a plurality of articulation zones 36 disposed between the insertion end 33 and a the bottom end 22 to enable the earplug 12 to be bent in a variety of directions so that it can better adapt to the contours of the ear canal—see U.S. Pat. No. 6,484,726 to Remer et al. for further discussion. Alternatively, the body portion 31 of the earplug 12′ can be configured in a cylindrical or bullet-like shape as shown in FIG. 7b. Each earplug 12, 12′ is arranged in each sheath 14 such that the body portion axis runs generally parallel to the longer dimension of the sheath 14. When the earplugs are configured to generally decrease in diameter going from end 22 to end 33, the sheath 14 may be tapered to generally correspond to such diameter decrease. The sheath 14 may be made from a deformable material that desirably is tear resistant and transparent.

As shown in FIG. 8, the sheath 14 exhibits these deformable, tear-resistant, and transparent qualities so that the earplug can be deformed by the user into a desired compressed condition before being inserted into the ear canal. The sheath 14 protects the earplug 12 from being exposed to contaminants that may be present on the user's fingers, while at the same time allowing for the earplug 12 to be compressed into a pre-insertion condition. The deformable nature of the sheath 14 allows the user to roll the foam earplug 12 between the user's opposable digits. Any contaminants that may be present on the user's fingers would only be transferred to the sheath 14 and not to the earplug 12. Thus, once the earplug 12 has been compressed into its desired pre-insertion condition, the ear plug 12 may be removed from the sheath 14. The user can grasp the end 22 of the compressed earplug 12 and insert the body portion 31 (FIG. 7a) into the ear canal before it expands to its generally relaxed condition. Once inserted into the ear canal, the earplug will slowly expand from the compressed condition to fill the ear canal so that it can properly attenuate noise that may be present in the ambient environment. The sheath preferably is transparent so that the user can visually witness the earplug deformation step. As the term is used in this document, “transparent” means capable of transmitting light sufficient for a person to at least generally see the object on the opposing side of the packaging material. As such, the term transparent includes films that would normally be considered translucent. For further discussion of an earplug disposed in a transparent deformable sheath, see applicants' copending U.S. patent application Ser. No. ______, entitled Foam Earplug in Deformable Sheath, filed on the same day as this patent application (attorney case number 63808US002).

Foam earplugs may be made from a variety of materials. The earplugs may be made from prepolymers, for example, isocyanate prepolymers, polypropylene glycol, tripropylene glycol, and polyols at various ratios. Additional materials such as surfactants, stabilizers, foaming agents, and water also may be used in making foam earplugs. The earplug forming composition may be poured into a mold, allowed to react and/or caused to rise to produce a foam earplug suitable for use in connection with the present invention. Rising may be accomplished by any suitable means, for example by using a self-rising foam, or by heating the mold. As the foam rises, it interpenetrates into the pores of the porous component, thereby forming a mechanical bond between the porous component and the finished foam. The earplugs may include a film, coating, or skin on an outer surface of the earplug. Such film may be continuous or discontinuous and may extend around the circumference of the earplug or exist in a discrete portion or region. Foam earplugs may contain a matrix of voids or cells within a solid polymeric material. The resulting foam earplugs may contain a variety of polymers, for example, polyvinyls such as PVC, polyurethanes, viscoelastic polymers, polyethers, and combinations thereof. Examples of polymeric foam earplugs are described, for example, in the following U.S. Pat. Nos. 6,920,956 to Falco, 6,484,726 to Remer et al., 6,105,715 to Knauer, 5,799,658 to Falco, 5,203,352 to Gardner, Jr., and D445,893 and D445,894 to Remer et al. Externally and internally plasticized polymeric foams disclosed in U.S. Pat. No. Re. 29,487 to Ross are generally suitable for use as a material of construction for a foam earplug. These plasticized polymeric foams are slow recovery foams that are not only comfortable but tend to deliver high-in-field noise protection at various hearing frequencies. U.S. Pat. No. 5,203,352 to Gardner discloses temperature-dependent viscoelastic polymeric foam materials, which comprise a slow recovery polymer and a faster recovery polymer. Another suitable foam is described in U.S. Pat. No. 5,420,381 to Gardner, Jr. Further, U.S. Pat. No. 5,792,998 to Gardner, Jr. et al. discloses a dynamically stiff foam material that has a low static stiffness and a high dynamic stiffness. The foam component can have a dynamic spring constant of at least about 300 pounds per inch and a dynamic loss factor of at least about 0.25. One particularly suitable dynamically stiff foam material is a polyurethane material. Additional suitable polyurethane foam formulations are disclosed in U.S. Pat. No. 4,158,087 to Wood. Commercially available foam earplugs include the 1100™ and 1110™ from the 3M Company.

Earplugs that may be used in accordance with the present invention may also include earplugs of the reusable variety. These earplugs typically comprise one or more flanges that extend from a central stalk. The stalk also may include a handle that allows the user to conveniently insert and remove the earplug from the ear canal. Examples of reusable earplugs are shown in the following U.S. Pat. D467,333 to Martinson; Des. 369,655, Des. 375,550, and Des. 375,551 to Esler et al.; Des. 427,304 to Magidson et al.; Des. 434,139 to Foslien; Des. 436,164 to Foslien; 4,936,411 to Leonard; and 6,241,042 to Falco. Commercially available reusable earplugs include the 1260™, 1270™, and 1271™ earplugs of the 3M Company.

One or two earplugs typically would be placed in each compartment of the package portion of the earplug assembly. If two earplugs are disposed in each compartment, they can be tethered together as disclosed in U.S. Pat. No. 4,193,396 to Wacker.

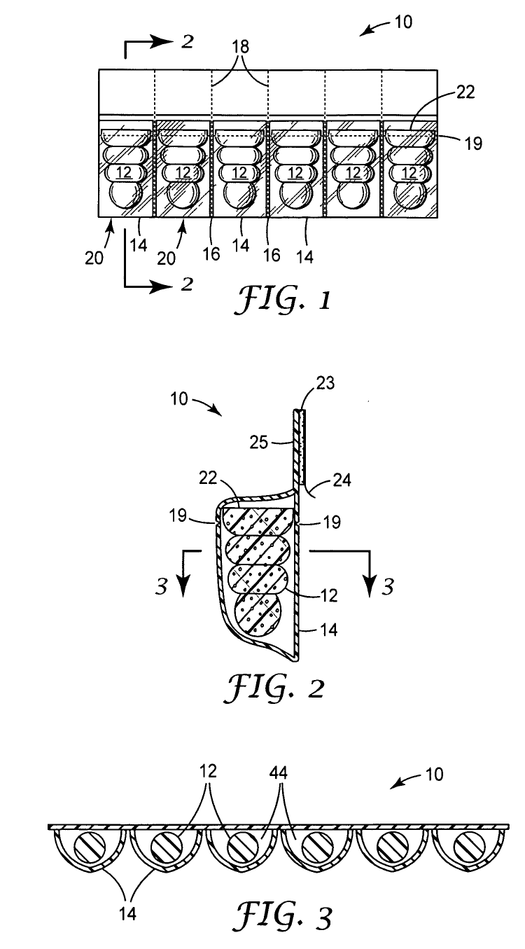

The packaging material that is used in connection with the present invention may comprise various films, sheets, foils, formed materials, plastic receptacles, cardboard packages. Such materials may include paper sheets, coated paper sheets, metallic foils, and a wide variety of polymeric materials. Polymeric materials may be extruded films, blown films, cast films, and films subjected to an orientation process. Rigid extruded polymeric materials also could be used. Polymers also can be formed into sheath structures using well-known processes such as injection molding, profile extrusion molding, and coating processes. The sheath material may be transparent so that the user can see the earplug to facilitate compression of a foam earplug. Transparency also can facilitate removal and insertion of the earplug from and to the sheath. The sheath also may comprise a polymeric film that contains one or more layers of polymeric materials. A polymeric sheath may be deformable but may also be resistant to being easily torn when subjected to shear forces from rolling and twisting between a user's fingers. Typically, a plastic sheath material will exhibit a tensile modulus of less than about 10 Giga Pascals (GPa) and greater than about 1 Mega Pascal (MPa); more typically a tensile modulus of less than about 1 GPa and greater than about 100 MPa. The sheath material typically has a thickness of about 0.2 mils (5 micrometers (μm)) to about 20 mils (508 μm), more typically, about 0.5 mils (13 μm) to about 10 mils (254 μm). The sheath volume typically is about 1 to 14 cubic centimeters (cm³), more typically about 2 to 12 cm³, and still more typically about 3 to 6 cm³. The sheath volume typically is about 5 to 200% greater than the earplug volume, more typically about 10 to 100% (or 15 to 50%) greater than the earplug volume. The sheath volume is measured as the volume of the whole sheath before being opened at separation line 19 (FIGS. 1, 2, 4, and 5). Polymeric sheaths may be made into a corrugated-type shape (see FIG. 3) using known procedures—see, for example, U.S. Pat. No. 5,961,778 to Braun. Other earplug receptacles are shown in U.S. Pat. Des. 434,139 and 436,164 to Foslien.

### EXAMPLES

### Example 1

An earplug assembly was created in the following manner. Two polymer film sheets were cut. The first piece was 2½ inches tall (6.35 cm) and 6 inches long (15.24 cm). The second piece was 1½ inches tall (3.81 cm) and 6 inches long (15.24 cm). The polymer film consisted of three layers: the center layer was an elastomeric material, and two outer layers were polypropylene. Both pieces were perforated using a rotary perforating tool approximately 1 inch (2.54 cm) from the bottom edge. Vertical welds were made, starting at the edge, at 1 inch intervals producing a series of six adjoining tubes.

The tubes were welded along the bottom edge to create individual pockets for the earplugs. A perforation was made along each of the vertical weld lines from the bottom edge to the horizontal perforation point. A 3M™ 1120 earplug was compressed and inserted into each pocket. A final weld was made along the top edge of the smaller piece of polyethylene to contain earplugs. A strip of transfer adhesive with release liner was applied to the back side near the top edge to create an earplug assembly similar to the assembly shown in FIG. 7.

### Example 2

An earplug assembly was prepared by cutting a sheet of a thin polymer film into a piece 5 inches long (12.7 cm) and 2 inches tall (5.08 cm). A second sheet was cut into a piece 9 inches (22.86 cm) long and 2 inches (5.08 cm) tall. The polymer film consisted of three layers: the center layer was an elastomeric material, and two outer layers were polypropylene. Each sheet was perforated, using a rotary perforating tool, in a horizontal line the entire length of each sheet, 1¼ inches (3.18 cm) from the bottom edge.

One side edge of the 5 inch (12.7 cm)×2 inch (5.1 cm) piece of material was aligned with one side edge of the 9 inch (22.9 cm)×2 inch (5.1 cm) piece of material and a ⅛ inch (3.2 mm) wide vertical heat weld was made attaching the edges together. The vertical weld extended approximately 1⅜ inches (3.5 cm). Eight additional vertical welds were made between the material pieces to form loops in the following manner. The 9 inch (22.9 cm)×2 inch (5.1 cm) piece of material was gathered at 1¼ inch (3.2 cm) intervals, placed on the 5 inch (12.7 cm)×2 inch (5.1 cm) piece of material at ⅝ inch (1.6 cm) intervals and welded. This procedure produced a looping structure of sheath pockets 44 as illustrated in FIG. 3 when viewed from the long edge.

3M™ 1120 Earplugs were first compressed and then inserted into each of the pockets until the grasping end 22 (FIG. 2) of the earplug was generally aligned with the perforation line 19. Another heat weld was made along the entire bottom edge to produce a package similar to the assembly shown in FIGS. 1 and 2.

A final heat weld was made perpendicular to the nine vertical weld lines, above the back ends of the earplugs to enclose the earplugs. A strip of transfer adhesive with release liner was attached to one side of the earplug system, across the length of the package, above the horizontal perforation line. The top of the package was folded between the weld line and the edge of the transfer adhesive to form a more compact package. Cuts were made down the center of each vertical weld from the bottom edge up to the perforation line.

The release liner was removed and the earplug assembly was attached to the side of a hard hat. A single earplug in a sheath was grabbed with one hand and pulled until it separated along the perforation line. The earplug was compressed while still contained in the sheath using thumb and fingers. The earplug was then removed from the sheath with the other hand and inserted in the ear using standard practice.

This invention may take on various modifications and alterations without departing from its spirit and scope. Accordingly, this invention is not limited to the above-described but is to be controlled by the limitations set forth in the following claims and any equivalents thereof.

This invention also may be suitably practiced in the absence of any element not specifically disclosed herein.

All patents and patent applications cited above, including those in the Background section, are incorporated by reference into this document in total. To the extent there is a conflict or discrepancy between the disclosure in such incorporated document and the above specification, the above specification will control.

What is claimed is:

1. An earplug assembly that comprises:
(a) one or more earplugs disposed in a package; and
(b) a means for securing the package to another object, the means for securing to another object being joined to the package.

2. The earplug assembly of claim 1, wherein the package comprises a deformable transparent plastic sheet.

3. The earplug assembly of claim 2, wherein the means for securing includes an adhesive strip that is joined to the package.

4. The earplug assembly of claim 3, further comprising a release liner that is juxtaposed against the adhesive strip.

5. The earplug assembly of claim 4, wherein the package is formed into a transparent plastic sheath that has a volume that is about 5 to 200% larger than a volume of one of the earplug(s).

6. The earplug assembly of claim 5, wherein the earplug(s) are disposed in the package and the adhesive strip is joined to thereto such that the package drapes beneath the adhesive strip when the assembly is secured to another object.

7. A combination, which comprises a helmet and the earplug assembly of claim 6 secured thereto.

8. A combination, which comprises a tool and an earplug assembly of claim 6 secured thereto.

9. The earplug assembly of claim 1, wherein one to two earplugs are disposed in the package.

10. The earplug assembly of claim 9, wherein each package contains only one earplug, and wherein the package is in the form of a plastic deformable sheath.

11. The earplug assembly of claim 1, comprising a plurality of packages that contain one or more earplugs.

12. The earplug assembly of claim 11 comprising up to 20 packages that contain one or more earplugs.

13. The earplug assembly of claim 12 comprising 2 to 10 packages that contain one or more earplugs and that are separated by a separation line.

14. The earplug assembly of claim 13, wherein the securing means includes an adhesive strip.

15. An earplug assembly that is in the form of a roll and that comprises 10 or more earplug packages that are capable of being separated from adjacent packages at a separation line, the packages comprising a deformable plastic sheet.

16. The earplug assembly of claim 15, wherein the roll includes an adhesive strip located along one edge of the roll.

17. The earplug assembly of claim 16, wherein the roll is provided in a dispenser.

18. The combination of claim 7, wherein the assembly comprises a plurality of deformable plastic packages that are capable of being separated from one another along a separation line.
